(19) United States
(12) Patent Application Publication
(10) Pub. No.: US 2007/0134318 A1
(43) Pub. Date: Jun. 14, 2007

(54) Non-tabletted, chewable, individually dosed administration forms

(76) Inventors: José Luis Fabregas Vidal, Barcelona (ES); Antoni Masso Carbonell, Barcelona (ES); Nuria Garcia Gonzalez, Barcelona (ES); Pere Guiro Coll, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/578,515

(22) PCT Filed: Nov. 9, 2004
(86) PCT No.: PCT/EP04/12658

§ 371 Date: May 8, 2006

(30) Foreign Application Priority Data
Nov. 10, 2003 (ES) P200302612

(51) Int. Cl. A61K 9/20

(52) U.S. Cl. 424/464

(57) ABSTRACT

Non-tabletted, individually dosed administration forms comprising a composition of at least one pharmaceutically active substance dissolved or dispersed within a matrix material comprising a mixture of at least 0.2% by weight of a gelatine, at least one stabilising agent and at least one water-soluble alcohol and/or water as a solvent, which composition is plastic at elevated temperature, characterised in that: the stabilising agent is chosen from (i) esters of glycerine and fatty acids; (ii) products resulting from the alcoholysis/esterification reaction of such esters of glycerine and fatty acids with polyethylenglycols; in that the stabilising agent has a melting point in the range of 42° C. to 63° C.; and in that water is present in an amount not greater than 46% by weight of the composition.

The invention relates to individually dosed administration forms for pharmaceutically active compounds, consisting of non-tabletted, chewable gel compositions packaged in blisters or cavities; to a process for the manufacture of such individually dosed administration forms; to individually dosed administration forms obtainable by the above-mentioned process; and to the use of a stabilising agent to enhance the ease of removal of the composition from the blisters or cavities.

Chewable delivery systems, such as chewing gums, are highly desirable means for the oral administration of pharmaceutically active compounds. A disadvantage of chewing gum compositions is that they generally include a water insoluble gum base, which remains in the mouth and must be disposed of. In addition, many active compounds may have affinity for the gum base, making thus accurate dosing difficult.

British Patent application GB 2 009 597 discloses chewable and swallowable, gelled antacid compositions. The compositions are obtained by dispersing an antacid in a solution comprising water, a carbohydrate or a polyhydric alcohol as a bodying agent and an amount of gelling agent sufficient to cause the liquid dispersion to set to a self-supporting gel after cooling. In a preferred embodiment the still liquid dispersion can be poured before cooling into oral unit dosage moulds and allowed to set.

This process no longer requires separate shaping and packaging of solid administration forms. These are given their particular shape during the packaging operation by simple application of the softened composition into a substrate with the desired shape, followed by solidification. This results in an improved cost efficiency of the overall manufacturing process.

International patent application WO 87/00429 describes opacified gelatine compositions and processes for their manufacture. The compositions comprise fats, fatty oils or fat derivatives to improve the light stability of the dyes used to colour the gelatine compositions. The specification states that all fats, fatty oils or fat derivatives of synthetic or natural origin, as well as partially hydrogenated products can be used, provided that they are physiologically safe.

It has now been found by the inventors that the use of gelatine as a gelling agent for the manufacture of non-tabletted, chewable compositions as those described in the prior art yields compositions that, upon ageing, do often present the problem that they cannot be easily removed from the packaging where they have been shaped without leaving residues in the packaging. The problem of residues left in the packaging upon removal of the jelly composition is particularly pronounced for compositions comprising a high amount of alkaline ingredients since these ingredients tend to destabilize the gelatine matrix. The problem is also particularly pronounced when the shape of the packaging shows edges or portions with a small radius of curvature.

The inventors have solved this problem by incorporating into a matrix material, comprising a mixture comprising a gelatine at least one water-soluble alcohol and/or water as a solvent and at least one stabilising agent selected from the group consisting of esters of glycerine and fatty acids and products resulting from the alcoholysis/esterification reaction of such esters of glycerine and fatty acids with polyethyleneglycols, the stabilising agent having a melting point in the range of 42° C. to 63° C. This results in non-tabletted, individually dosed administration forms comprising a composition of at least one pharmaceutically active substance dissolved or dispersed within the matrix material, which composition is plastic at elevated temperature. These administration forms can be removed from the packaging without leaving residues. In a preferred embodiment of the present invention only one stabilising agent is incorporated into the matrix material.

As essential ingredients the composition of the present invention comprises at least one pharmaceutically active substance, gelatine present in an amount of at least 0.2% by weight of the composition, at least one stabilising agent as described above, and at least one water-soluble alcohol and/or water as a solvent, wherein water is present in an amount not greater than 46% by weight of the composition. It may also comprise bodying agents that impart texture and body to the final gel, and other optional components such as preservatives, antioxidants, defoaming agents, sweeteners, taste-masking agents, colour and flavours. It is a preferred embodiment of the present invention that only one stabilising agent is used.

The bodying agents suitable for the present invention are sugars such as glucose, sucrose and fructose, sugar alcohols such as sorbitol, mannitol and maltitol and polysaccharides such as starch, cellulose and functionalised cellulose derivatives.

To ensure consumer acceptability it is preferred that the non-tabletted, individually dosed administration forms of the present invention have compositions showing no plastic deformation at temperatures below 37° C.

Gelatine is a protein obtained by extraction from animal raw materials containing collagen such as skins and bones, which have been previously conditioned by acidic or alkaline treatment. Commercially available gelatine typically contains 84-92% protein, 0.1-2% salts and the rest is water.

Commercially available gelatines are classified according to the raw material from which they have been obtained and according to their ability to gel, which is customarily measured as Bloom gel strength.

Although all types of gelatine can be used for the manufacture of the individually dosed administration forms of the present invention, it has been found that gelatines with a Bloom range comprised between 140 and 270 degrees Bloom, preferably between 180 and 250 degrees Bloom yield composition with optimum consumer acceptance in terms of palatability. Gelatines obtained though alkaline treatment are in general preferred to those obtained through acidic treatment.

It is preferred that the compositions of the present invention comprise gelatine in an amount greater than 0.2% by weight of the composition, more preferably greater than 1% by weight and still more preferably greater than 5% by weight of the composition.

The stabilising agent of the present invention is selected from the group consisting of esters of glycerine and fatty acids and products resulting from the alcoholysis/esterification reaction of such esters of glycerine and fatty acids with polyethyleneglycols having a melting point in the range of 42° C. to 63° C.

Examples of such stabilising agents are the mono-, di- and triesters of glycerine with fatty acids and mixtures thereof, preferably the diesters of glycerine with fatty acids. Preferred fatty acids are those selected from C10-C20, preferably C16-C18, unsaturated, saturated fatty acids. Examples of such fatty acids are lauric, oleic, linoleic, linolenic, palmitic and stearic acids. An example of a preferred commercially available ester is Estol® 3745 GDS T2 from Uniqema. Other examples of stabilising agents are the products of the alcoholysis/esterification reaction of the esters of glycerine and fatty acids mentioned above. Preferred examples are products of the alcoholysis/esterification reaction of hydrogenated palm kernel oil or hydrogenated palm oil with PEG 1500, such as Gelucire® 44/14 and Gelucire® 50/13 from Gattefossé.

In an embodiment of the invention the stabiliser is present in an amount greater than 1% by weight of the formulation.

In an embodiment of the invention the solvent or solvents present in the composition is/are used in a total amount of at least 10% by weight, more preferably greater than 25% by weight still more preferably greater than 50% by weight of the composition.

In a preferred embodiment of the invention the composition comprises more than 46% by weight of the composition of at least one water-soluble alcohol.

In another embodiment of the present invention the amount of water of the present compositions is not greater than 46% by weight, preferably not greater than 35% by weight, most preferably not greater than 25% by weight, most preferably not greater than 15% by weight of the composition.

The compositions of the present invention comprise at least one pharmaceutically active substance which is dispersed or dissolved within the matrix material when it is in the molten state. The pharmaceutically active substance need not be in any specific form for its successful incorporation within the molten matrix material, in particular it is not required, and also not preferred, that the pharmaceutically active substance is provided as a component of a shearform matrix carrier prepared by flashflow processing.

In an embodiment of the present invention the non-tabletted, individually dosed administration forms comprise more than 18% by weight of a pharmaceutically active substance.

Suitable pharmaceutically active substances that may be contained in the individually dosed administration forms of the present invention vary widely and generally represent any stable drug combination. Illustrative categories and specific examples include:
a) ANTACIDS:
   i) Inorganic or organic salts of aluminium, for example, aluminium allantoinate, aluminium aminoacetate, aluminium phosphate, aluminium silicate, aluminium glucoheptanoate or aluminium polygalacturonate.
   ii) Inorganic or organic salts of bismuth, for example, bismuth aluminate, bismuth carbonate, bismuth silicate, bismuth subcarbonate or bismuth citrate.
   iii) Inorganic or organic salts of calcium, for example, calcium phosphate or calcium aminoacetate.
   iv) Inorganic or organic salts of magnesium, for example, magnesium carbonate, basic magnesium carbonate, magnesium phosphate or magnesium silicate.
   v) Oxides and hydroxides, such as aluminium oxide, algeldrate (aluminium hydroxide), magnesium or calcium oxides or hydroxides.
   vi) Mixed salts of aluminium and sodium as silicate, mixed salts of aluminium and magnesium as hydrotalcite (basic aluminium and magnesium carbonate), almagate (basic aluminium and magnesium carbonate) or magald rate (basic aluminium and magnesium sulphate), mixed salts of bismuth and magnesium as magnesium silicate, and magnesium aluminosilicates, as simaldrate or almasilate.
   vii) Hydrogen carbonates assodium or potassium hydrogen carbonates.
   viii) Glycine.
   ix) Alginic acid and salts thereof.
   and mixtures thereof.
b) DRUGS FOR PEPTIC ULCER AND GASTRO-OESOPHAGEAL REFLUX
Ranitidine*, Nizatidine, Famotidine, Cimetidine, Roxatidine, Pifatidine, Roxatidine, Sufotidine, Lafutidine, Osutidine, Pantoprazole, Omeprazole, Lansoprazole, Esomeprazole, Rabeprazole, Esaprazole, Pariprazole, Aripiprazole, Leminoprazole, Amoxicillin, Trospectomycin, Clarithromycin, Zinc Acexamate, Cetraxate, Rotraxate, Dosmalfate, Flavalfate, Sucralfate, Bismuth salts as bismuth citrate or subsalicylate, Triletide, Dicloguamine, Sulfoxazine, Rioprostil, Ritipenem, Trimoprostil, Benexate, Pramipide, Misoprostol, Alaptide, Proglumide, Azuletil, Trepenone, Polyenephosphatidylcholine, Plaunotol, Troxipide, Midoriamine, Ecabet, Quinotolast, Sulglicotide, Nitazoxanide, Revaprazan, and mixtures thereof.
c) DRUGS FOR FUNCTIONAL GASTROINTESTINAL DISORDERS; PROPULSIVES
Metoclopiamide, Cinitapride, Clebopride, Cisapride, Zacopride, Mosapride, Itopride, Prucalopride, Domperidone, Ecabapide, Polycarbophil calcium, Tegaserod, and mixtures thereof.
d) LAXATIVES
Sennatin, Sennosides A+B, Glycerol, Picosulfate, Lactitol, Bisacodyl, Polyethylene glycol, Lactulose, Basic magnesium carbonate, and mixtures thereof.
e) ANTIOBESITY PRODUCTS
Orlistat, Amfebutamone, Bupropion, Diethylpropion, Sibutramine, Fluoxetine, Metaraminol, Mazindol, Chorionic gonadotrophin, Phentermine, Metamfetamine, Phendimetrazine, Benzfetamine, Phenylpropanolamine, Fenproporex, and mixtures thereof.
f) DIGESTIVES; ENZIME PREPARATIONS
Amilase, Cellulase, Lactase, Lipase, and mixtures thereof.
g) VITAMINES
Mixtures of vitamines, mixtures of oligoelements, and mixtures thereof.
h) APPETITE STIMULANTS
Pizotifen, Cryptoheptadine, Carnitine, Stolimine, and mixtures thereof.
i) ANTITHROMBOTIC AGENTS; PLATELET AGGREGATION INHIBITORS
Ditazole, Acetylsalicylic acid, Trifusal, Epoprostenol, Eptifibatide, Heparin, Clopidrogel, Dipyridamole, Abciximab, Ticlopirine, Dalteparin, Danaparoid, Warfarin, Phenindione, Dicoumarol, Epoprostenol, Enoxaparin, Nadroparin, Antithrombin III, Indobufen, Parnaparin, Tinzaparin, Dermatan, Desirudin, Reviparin, Thombomoduline, Bivalirudin, Ardeparin, Lepirudin, Tifacogin, Fondaparine, Fenprocumone, Certoparin, Bemiparin, Idraparinux, Acenocoumarol, Gabexate, Sulodexide, Defibrotide, Isbogrel, Cilostazol, Ciprostene, Ataprost, Sulotroban, Taprostene, Cloricromen, Picotamide, Alprostadil, Sulfinpyrazone, Beraprost, Daltroban, Variprost, Satrigel, Sarpogrelate, Tirofiban, Ecraprost, Lamifiban, Lefradafiban, Xemilofiban, Polycosinol, Roxifiban, Lotrafiban, Sibrafiban, Alnidofibatide, Orbofiban, Argatroban, Ticlomarol, and mixtures thereof.
j) ANTIANEMIC PREPARATIONS; TRIVALENT IRON PREPARATIONS
Ferritine, Ferric proteine succinate, Ferric dextran and mixtures thereof.
k) ANTIARRHYTHMICS
Quinidine, Esmolol, Pirmenol, Acecainide, Pilsicainide, Recainam, Penticainide, Flecainide, Adenosine, Lidocain, Metoprolol, Propranolol, Nadolol, Oxprenolol, Phenytoin, Acebutolol, Sotalol, Carteolol, Medigoxine, Procainamide, Bretylium, Amiodarone, Disopyramide, Mexiletine, Moracizine, Tocainide, Propafenone, Barucainide, Alprenolol, Otenzepad, Verapamil, Diprafenone, Etacizin, Bidisomide, Arotinolol, Cibenzoline, Tiracizine, Pindolol, Diltiazem, Atenolol, Dofetilide, Tedisamil, Sematilide, Sotalol, Almokalant, Nifekalant, Ibutilide, Landiolol, Dronedarone, Talinolol, Tecadenoson, Digoxin, Indenolol, Prajmalium, Aprindine, Bunaftine, Butobendine, Lorajmine, Lorcainide, and mixtures thereof.
l) CARDIAC STIMULANTS, ORGANIC NITRATES
Isosorbide mononitrate or dinitrate, Nitroglycerol, Pentaerythrityl tetranitrate, Molsidomine, and mixtures thereof.
m) ANTI HYPERTENSIVES; ALPHA ADRENORECEPTOR ANTAGONISTS
Doxazosin, Urapidil, Nipradilol, Indoramin, Prazosin, Labetalol, Amosulalol, Terazosin, Monatepil, and mixtures thereof.
n) DIURETICS
Triamterene, Canrenoate, Spironolactone, Furosemide, Torasemide, Cicletanine, Piretanide, Chlorothiazide, Chlortalidone, Hydroflumethiazide, Bendroflumethiazide, Methyclothiazide, Polythiazide, Clopamide, Quinethazone, Bumetanide, Indapamide, Xipamide, Cyclopenthiazide, Canrenone, Docarpamine, Hydrochlorothiazide, Metolazone, Azosemide, Anaritide, Ularitide, Ecadotril, Candoxatril, Amiloride, Ethacrynic acid, Conivaptan, Telmisartan, Mebutizide, and mixtures thereof.
o) PERIPHERAL VASODILATORS
Dihydroergocristine, Piracetam, Nioergoline, Vinburnine, Cadralazine, Flunarizine, Metergoline, Hydralazine, Fasudil, Nicorandil, Linsidomine, Sildenafil, Cinnarizine, Heptaminol, Almitrine, Raubasine, Pentoxifyline, Trimetazidine, Buflomedil, Alprostadil, Brovincamine, Cinepazet, Dilazep, Lidoflazine, Molsidomine, Nicorandil, Nifedipine, Trapidil, Viskenit, and mixtures thereof.
p) VASOPROTECTIVES
Diosmin, Hidroxmin, Hesperidin, Troxerutin, and mixtures thereof.
q) ANTI HYPERTENSIVES—SELECTIVE BETA BLOCKING AGENTS
Atenolol, Esmolol, Carteolol, Metoprolol, Bisoprolol, Carvedilol, Nebivolol, Propranolol, Tertatolol, Betaxolol, Cetamolol, Nipradilol, Tilisolol, Mepindolol, Nadolol, Oxprenolol, Acebutolol, Sotalol, Timolol, Labetalol, Penbutolol, Celiprolol, Amosulalol, Alprenolol, Cloranolol, Bopindolol, Soquinolol, Arotinolol, Pindolol, Talinolol, Esatenolol, Indenolol, Befunolol, Bevantolol, Bucomolome, Bunitrolol, Butofilolol, Carazolol, Lervonoprolol, Nifenalol, Rescimetol, Bunazosin, Doxazosin, Guanabenz, Guanadrel, Guanfacine, Guanoxabenz, Indoramine, Rilmenidine, Lofexidine, Naftopidil, Prazosin, and mixtures thereof.
r) SELECTIVE CALCIUM CHANNEL BLOCKERS WITH MAINLY VASCULAR EFFECTS
Amlodipine, Nisoldipine, Nicardipine, Nitrendipine, Felodipine, Anipamil, Zonisamide, Benidipine, Darodipine, Tiapamil, Tetrandrine, Lercanidipine, Gallopamil, Bepridil, Diproteverine, Isradipine, Franidipine, Nivaldipine, Levetiracetam, Nimodipine, Verapamil, Aranidipine, Fasudil, Dotarizine, Lacidipine, Lomerizine, Cilnidipine, Nifedipine, Diltiazem, Palonidipine, Monatepil, Fantofarone, Semotiadil, Efenidipino, Manidipine, Barnidipine, Elgodipine, Pranidipine, Furaldipine, Ciclandelate, and mixtures thereof.
s) AGENTS ACTING ON THE RENIN-ANGIOTENSIN SYSTEM; ACE INHIBITORS
Enalapril, Ramipril, Quinapril, Captopril, Perindopril, Fosinopril, Trandolapril, Cilazapril, Lisinopril, Spirapril, Moexipril, Delapril, Alacepril, Enalaprilat, Benazepril, Fentiaprii, Zofenopril, Fosinoprilat, Utibapril, Temocapril, Ceranapril, Zofenoprilat, Imidapril, and mixtures thereof.
t) ANGIOTENSIN II ANTAGONISTS
Candesartan, Losartan, Eprosartan, Irbesartan, Valsartan, Tasosartan, Telmisartan, Olmesartan, and mixtures thereof.
u) CHOLESTEROL AND TRIGLYCERIDE REDUCERS
Atorvastatin, Lovastatin, Eptastatin, Simvastatin, Fluvastatin, Dalvastatin, Itavastatin, Rosuvastatin, Pravastatin, Probucol, Polycosanol, Ciprofibrate, Fenofibrate, Benzafibrate, Clofibrate, Filicol, Gemfibrozil, Benfluorex, Colestyramine, Phytosterols, Acipimox, Binifibrate, Clinofibrate, Colestilan, Diethylaminoethyl Dextran, Colestrol, Etiroxate, Etofibrate, Gugulipid, Meglutol, Melinamide, Niceritrol, Omacor, Pirifibrate, Sorbinicate, Sulodexide, Sultosilic Acid, and mixtures thereof.
v) ESTROGENS; FEMALE CONTRACEPTIVES
Estradiol, Ethinylestradiol, Norethisterone, and mixtures thereof.
w) DRUGS USED IN BENIGN PROSTATIC HYPERTROPHY
Pygeum Extract, Alfuzosin, Dutasteride, Finisteride, Oxendolone, Tamsulosin, and mixtures thereof.
x) CALCIUM HOMEOSTASIS; ANTIPARATHYROID HORMONES
Calcitonin, Elcatonin, and mixtures thereof.
y) ANTINEOPLASTIC AGENTS
Ameticine, Atrimustine, Diaziquone, Spiromustine, Melphalan, Elmustine, Estramustine, Ranimustine, Dibromomulcitol, Tauromustine, Temozolomide, Carboplatin, Fotemustine, Aranose, Perfosfamide, Eptaplatin, Busulfan, Porfiromycin, Ifosfamide, Clorambucil, Altretamine, Cisplatin, Lomustine, Improsulfan, Mitobronitol, Mitolactol, Nedaplatin, Oxaliplatin, Prednimustine, Temozolomide, Treosuflan, Trofosfamide, Cyclophosphamide, Methotrexate, Butocin, Capecitabine, Carmofur, Cladribine, Cytarabine, Doxifluridine, Enocitabine, Fludarabine, Gemcitabine, Pentostatin, Raltitrexed, Tegafur, Etoposide, Pirarubicin, Aminoglutethimide, Anastrozole, Bicalutamide, Clodronate, Epitiostanol, Exemestane, Fadrozole, Flutamide, Formestane, Fulvestrant, Letrozole, Mepitiostane, Nilutamide, Tamoxifen, Toremifene, Trilostane, Krestin, Lentinan, Picibanil, Procodazole, Sizofuran, Ukrain, Virulizin, Alitretinoin, Amsacrine, Bexarotene, Docetaxel, Irinotecan, Miltefosine, Mitoxantrone, Nitracrine, Bortezomib, Paclitaxel, Porfimer, Razoxane, Sobuzoxane, Teniposide, Topotecan, Vindesine, Vinorelbine, Geftinib, Imatinib, Bleomycin, Megestrol, Lenograstim, and mixtures thereof.
z) ANTI INFLAMMATORY AND ANTIRHEUMATIC PRODUCTS
Aceclofenac, Diclofenac, Ketorolac, Meloxicam, Naproxen, Piketoprofen, Acemetacin, Alclofenac, Amfenac, Ampiroxicam, Azapropazone, Bufexamac, Butibufen, Carprofen, Chondroitin, Cinmetacin, Clidanac, Dexketoprofen, Diphenpyramide, Droxicam, Emorfazone, Enfenamic Acid, Epirizole, Etersalate, Fenbufen, Fentiazac, Feprazone, Flunoxaprofen, Flurbiprofen, Guaimesal, Ibuproxam, Indometacin, Ketoprofen, Lonazolac, Mabuprofen, Nabumetone, Nimesulide, Oxametacin, Parsalmide, Perisoxal, Piroxicam, Pranoprofen, Proglumetacin, Proquazone, Proticinic acid, Sulindac, Talniflumate, Tolfenamic Acid, Tolmetin, Zaltoprofen, Benzydamine, Etofenamate, Felbinac, Fepradinol, Idocrilamide, Loteprednol, Vessiflex, Glucosaline, Celecoxib, Hyaluronic Acid, Meclofenamate, Piproxen, Tenoxicam, Valdecoxib, Etoricoxib, Rofecoxib, and mixtures thereof.
aa) BISPHOSPHONATES
Risedronate, Tiludronate, Clodronate, Pamidronate, Etidronate, Alendronate, Zoledronate, Cimadronate, Neridronate, Olpadronate, Minodronate, Ibandronate, and mixtures thereof.
bb) ANALGESICS
Acetylsalicylic Acid, Paraoetamol, Codeine, Dihydrocodeine, Dexibuprofen, Alminoprofen, Carbasalate, Desflurane, Diflunisal, Enflurane, Etomidate, Floctafenine, Fosfosal, Isoflurane, Isonixin, Ketorolac, Lornoxicam, Clonixinate, Midazolam, Mofezolac, Naproxen, Nefopam, Propofol, Rimazolium, Rofecoxib, Ropivacaine, Sevoflurane, Parecoxib, Propacetamol, Zaltoprofen, Acemetacin, Sulindac, Indometacin, Mefenamic Acid, Ketoprofen, Diclofenac, Piroxicam, Flupirtine, Mofezolac, Ibuprofen, Fenoprofen, Flurbiprofen, Amtolmentin, Fepradinol, Celecoxib, Valdecoxib, Etoricoxib, Fluproquazon, Nefopam, Asthaxantin, and mixtures thereof.
cc) ANTI MIGRAINE PREPARATIONS
Almotriptan, Propofol, Gabapentin, Zonisamide, Lisinopril, Valproate, Pirprofen, Indoramin, Lidocain, Metoprolol, Ergotamine, Cyproheptadine, Propranolol, Pizotifen, Flunarizine, Nadolol, Metergoline, Ketoprofen, Methysergide, Buclizine, Timolol, Tiaspirone, Topiramate, Somatostatin, Etiracetam, Cinnarizine, Dihydroergotamine, Feverfew, Dronabinol, Dotarizine, Lomerizine, Ibuprofen, Sumatriptan, Naratriptan, Donepezil, Zolmatriptan, Naproxen, Rizatriptan, Montelukast, Frovatriptan, Botulinum Toxin, Alniditan, Avitriptan, Eletriptan, Metoclopramide, Targinine, Aminophylline, Tolfenamic Acid, Isometheptene, and mixtures thereof.
dd) ANTIEPILEPTICS
Phenobarbital, Clonazepam, Felbamate, Fosphentoin, Gabapentin, Lamotrigine, Levetiracetam, Oxcarbazepine, Tiagabine, Topiramate, Valproate, Vigabatrin, Zonisamide, Milaoemide, Denzimol, Bretazenil, Eterobarb, Diazepam, Chlormethiazole, Clonazepam, Clobazam, Mefobarbital, Mephenytoin, Primidone, Aoetazolamide, Valpromide, Ralitoline, Fengabine, Licarbazepine, Lorazepam, Antiepilepsirine, Rufinamide, Zaleplon, Abecamil, Losigamone, Selfotel, Midafotel, Remaoemide, Carbamazepine, Ethosuximide, Metsuximide, Retigabine, Valnoctamide, and mixtures thereof.
ee) ANTIPSYCHOYTICS
Haloperidol, Sulpiride, Blonanserin, Spiperone, Rimcazole, Isofloxythepin, Remoxipride, Emonapride, Bretazenil, Zuclopenthixol, Veralipride, Bromperidol, Droperidol, Trifluoperazine, Bromazepam, Levopromazine, Fluopromazine, Perphenazine, Thioridazine, Chlorprothixene, Fluphenazine, Periciazine, Tiotixene, Flupentixol, Benperidol, Fluspirilene, Pimozide, Clozapine, Pipotiazine, Loxapine, Tiapride, Zotepine, Sultopride, Lithium Carbonate, Asenapine, Tiaspirone, Ritanserin, Tandospirone, Amperozide, Clospipramine, Nalmefene, Prochlorperazine, Amisuipride, Levosulpriride, Risperidone, Promazine, Perospirone, Aripiprazole, Chlorpromazine, Carpipramine, Iloperidone, Remoxepride, Carbamazepine, Olanzapine, Quetiapine, Ziprasidone, Valproate, Azaperone, Cyamemazine, Timiperone, Bifeprunox, and mixtures thereof.
ff) ANXIOLYTICS
Diazepam, Clorazepate, Pyridoxine, Sulpiride, Lorazepam, Phenobarbital, Meprobamate, Buspirone, Suriclone, Citalopram, Brotizolam, Adinazolam, Etizolam, Bretazenil, Medicar, Enciprazine, Loflazepate, Propranolol, Chlordiazepoxide, Hydroxyzine, Trifluoperazine, Oxazepam, Medazepam, Clonazepam, Oxprenolol, Bromazepam, Clobazam, Nordazepam, Ketazolam, Halazepam, Alprozolam, Fluphenazine, Chlorimipramine, Venlafaxine, Ritanserin, Ipsapirone, Tandospirone, Buspirone, Pazinaclone, Flesinoxan, Fluoxetine, Selfotel, Zatosetron, Pagoclone, Carpipramine, Sunepitron, Sertraline, Paroxetine, Cyclobenzaprine, Cyamemazine, Valnoctamide, Clotiazepam, and mixtures thereof.
gg) ANTIDEPRESSANTS
Citalopram, Venlafaxine, Atomoxetine, Clopradone, Binedaline, Sertraline, Femoxetine, Oxaprotiline, Viqualine, Clovoxamine, Milacemide, Brofaromine, Cianopramine, Moclobemide, Midalcipran, Adinazolam, Nefazodone, Azamianserin, Reboxetine, Tianeptine, Toloxatone, Fluvoxamine, Amitriptyline, Imipramine, Trifluoperazine, Phenelzine, Fluphenazine, Flupentixol, Isocarboxazid, Tranylcypromine, Trimipramine, Desipramine, Opipramol, Nortriptyline, Protriptyline, Doxepin, Lithium Carbonate, Chlorimipramine, Dosulepin, Trazodone, Butriptyline, Viloxazine, Maprotiline, Amoxapine, Lofepramine, Bupropion, Ritanserin, Doconexent, Paroxetine, Ipsapirone, Fengabine, Tandospirone, Setiptiline, Amfebutamone, Lazabemide, Flesinoxan, Adrafinil, Ademetionine, Modafinil, Litoxetine, Fluoxetine, Ceronapril, Cericlamine, Beloxepin, Sunepitron, Agomelatine, Aprepitant, Amineptine, Nomifensine, Chromium Picolinate, and mixtures thereof.
hh) TREATMENT OF ALCOHOL DEPENDENCE
Acamprosate, Vigabatrin, Diazepam, Disulfiram, Ritanserin, Naltrexon, Nalmefene, Carbamazepine, Hydroxybutyrate, Nitrefazole, Metadoxine, and mixtures thereof.
ii) NASAL DECONGESTANTS
Pseudoephedrine, Fluticasone, Indanazoline, Tinazoline, Ipratropium, and mixtures thereof.
jj) DRUGS FOR ASTHMA/OBSTRUCTIVE AIRWAYS DISEASES
Salmeterol, Fenoterol, Ipratropium, Fluticasone, Beclometasone, Flutropium, Talniflumate, Terbutaline, Oxitropium, Rolipram, Seratrodast, Praniukast, Formoterol, Albuterol, Salbutamol, Midesteine, Tiotropium, Sibenadet, Roflumilast, Aminophylline, Budesonide, Almitrine, Glycopyrrolate, Bambuterol, Mabuterol, Procaterol, Tulobuterol, Rimiterol, Reproterol, Pirbuterol, Daltroban, Ramatroban, Tomelukast, Ibudilast, Pobilukast, Zafirlukast, Montelukast, Methylprednisolone, Dexamethasone, Triamcinolone, Tipredane, Mometasone, Loteprednol, Flunisolide, Hydrocortisone and mixtures thereof.
kk) EXPECTORANTS OR COUGH SUPPRESSANTS
Carbocisteine, Citiolone, Dropropizine, Cloperastine, Ozagrel, Nesosteine, Levodropropizine, Cistinexine, Dextromethorphan, Guaimesal, Nepinalone, Fudosteine, Quinidine, Hydrocodone, Noscapine, Chlorpheniramine and mixtures thereof.
ll) ANTIHISTAMINES FOR SYSTEMIC USE
Terfenadine, Ebastine, Dexchlorpheniramine, Azelastine, Acrivastine, Emedastine, Loratadine, Picumast, Diphenhydramine, Promethazine, Fenclozine, Levocabastine, Desloratadine, Cinnarizine, Setastine, Tagorizine, Mizolastine, Cetirizine, Tazifylline, Epinastine, Olopatadine, Bepotastine, Rupatadine, Norastemizol, Triprolidine, Fexofenadine, Ketotifen, Azatadine, Clemastine, Brompheniramine, and mixtures thereof.
mm) BUCAL ANTISEPTICS
Chlorhexidine, Chloramine-T, Benzalkonium Chloride, and mixtures thereof.
nn) OTHERS
Sulfamethoxazole, Centella, Calcium Folinate, Palmidrol, Thiomucase, Glucomannan, Leucocianidol, Bacterial Lysate, Spagul, and mixtures thereof.

It is preferred that active substance which can be present in the compositions according to the invention is selected from the group consisting of non-lipophilic active substances. The preferred pharmaceutically active substances are antacid compounds. The preferred antacids for use in the invention are generally carbonate or hydroxycarbonate salts of calcium, magnesium, aluminium, or bismuth and combinations thereof, and are generally very water insoluble. Other antacids such as sodium bicarbonate, calcium bicarbonate, and other carbonates, silicates, and phosphates are included in this invention. Preferred antacids are aluminium and magnesium antacids, such as, for example, aluminium hydroxide and magnesium hydroxide and also preferred are crystalline aluminium magnesium hydroxycarbonates or sulphates such as hydrotalcite, magaldrate and almagate. Almagate is particularly preferred. Mixtures of antacid compounds may be used if desired. When antiacids are used as pharmaceutically active substances they are present in amounts ranging from 5 to 50% by weight of the composition, preferably, between 10 and 45% by weight of the composition, more preferably between 20 and 35% by weight of the composition.

The compositions of the present invention preferably comprise water, more preferably at least 1% wt. water, and do not comprise edible gums.

The present invention relates also to a process for producing non-tabletted, individually dosed administration forms comprising the steps of: (a) forming a composition comprising at least one pharmaceutically active substance dispersed or dissolved within a matrix material comprising a mixture of gelatine, at least one stabilising agent and at least one water-soluble alcohol and/or water as a solvent, which is plastic at elevated temperature, and keeping such composition above 37° C. in a heating tank, (b) transferring the composition, when it is fluid into a heated dosing apparatus, (c) discharging the composition onto a shaped substrate, through a controlled mechanism so that a constant quantity of the fluid formulation material is thereby dosed onto the substrate, (d) cooling the composition, wherein the stabilising agent or agents present in the composition is/are selected from the group consisting of esters of glycerine and fatty acids and products resulting from the alcoholysis/esterification reaction of such esters with polyethyleneglycols and has a melting point in the range of 42° C. to 63° C.; and (e) optionally sealing the substrate containing the composition.

It is an optional embodiment of the present invention that an adhesion-reducing separating agent is placed on the inner surface of a cavity or a blister prior to step (c) of the above-mentioned process. Examples of such adhesion-reducing separating agents are lecithin, talc, starch, vaseline, and fats which are fluid at 25° C.

It is also a preferred embodiment of the present invention that the cavities or blister of the individually dosed administration forms are made of a material selected from PVC (polyvinyl chloride), PVDC (polyvinylidene chloride), PP (polypropylene), Aclar or laminates such as OPA-Aluminium-PVC (oriented polyamide-aluminium-polyvinyl chloride). PVC is particularly preferred. (in full)

The manufacturing processes described and claimed in European patent application number 0 250 578, which are explicitly incorporated by reference, are modified by the addition of the stabilising agent to the composition to be processed and constitute in this modified form particular preferred embodiments of the process under the present invention.

In another aspect the present invention relates to the use of at least one stabilising agent selected from the group consisting of (i) esters of glycerine and fatty acids (ii) products resulting from the alcoholysis/esterification reaction of such esters with polyethyleneglycols, and having a melting point in the range of 42° C. to 63° C. to facilitate the removal from the blisters or cavities where they have been packaged, of compositions comprising pharmaceutically active substances dispersed or dissolved within a matrix material comprising a mixture of gelatine and at least one water-soluble alcohol and/or water as a solvent, which composition is plastic at elevated temperature.

As used herein the term “plastic at elevated temperature” is meant to designate a composition which can be molded at temperatures comprised between 45° C. and 120° C. and keeps its molded shape after it cools to 20° C.

As used herein “melting point” is meant to designate the temperature at which the very last visible particle of a small substance's column introduced in a capillary melts as described in the European Pharmacopea 2.2.14. A suitable apparatus for this determination is the Melting Point Apparatus B-540 available from Büchi Labortechnik AG.

As used herein the term “non-tabletted administration form” is intended to mean any form which has not been manufactured by using conventional tabletting processes such as the tabletting of granular or powdery compositions in an exoentric or rotary press machine.

As used herein the term “edible gum” is intended to mean polysaccharide gums comprising among others gum arabic, gum tragacanth, agar agar, xanthan gum, alginates.

As used herein the term “water-soluble alcohol” is meant to designate a pharmaceutically acceptable, liquid monohydric or polyhydric alcohol which can be mixed with water to form a uniform solution in a quantity of at least 10 volumes of alcohol per 100 volumes of water. Examples of such alcohols are ethanol, n-propanol, iso-propanol, glycerol, propylene glycol, 1,3-butylene glycol and polyethylene glycols having a molecular weight comprised between 100 and 600 Dalton.

Removal from Blister Test

The compositions to be tested are manufactured according to the process described in example 1 and dosed into cylindrical cavities of circular cross-section having a diameter of 25 mm of a blister packaging made of PVC. The blister is thermo-sealed with an aluminium foil.

The blisters are then stored in a climatic chamber at 40° C. and 75% relative humidity for 10 weeks. After this period they are left at 25° C. and 60% relative humidity for 24 hours.

For each product to be tested a panel consisting of 5 expert panellists is given 5 samples of the formulation each, and the panellists are asked to remove the composition from the blister where it is packaged by pressing with the thumb on the plastic wall of the cavity until the composition is expelled from the cavity through the aluminium foil. After the composition has been expelled the remaining aluminium sealing film is removed and the plastic cavity is visually inspected. The panellist are asked to give a sample the rating “Failed” if residues exceeding 0,5 mm in any dimension can be seen in the empty cavity. Otherwise the rating “Passed” must be assigned.

### EXAMPLES

### Example 1

2060.8 g of a 85% solution of glycerine in water are heated in an Erweka SG3W reactor to 65-75° C. 288 g of pig skin gelatine of 240 degrees Bloom are slowly and continuously added during approximately 4 minutes until complete solubilisation has taken place. The mixture is stirred for 10 additional minutes. 48 g of lecithin are incorporated and the mixture stirred for 10 minutes. 800 g of almagate are then slowly and continuously added during approximately 15 minutes and the mixture stirred for 20 additional minutes at 75-80° C. 3.2 gr of flavour are successively incorporated and the solution stirred for 5 minutes. 4 g of the molten composition are dosed into the cylindrical cavities of circular cross-section having a diameter of 25 mm of a blister packaging made of PVC. The blister is thermo-sealed with an aluminum foil.

The composition of each individual cavity is as follows:

| Ingredient | % wt. |
|---|---|
| Almagate | 25.00 |
| Gelatine | 9.00 |
| Glycerine (100%) | 54.74 |
| Water | 9.66 |
| Lecithin | 1.50 |
| Flavour | 0.10 |

### Examples 2 to 7

Compositions 2 to 7 were manufactured following the process described in example 1 modified in that 1900.8 gr of the glycerine solution were used, and in that 160 g. of a stabilising agent were added after the complete solubilisation of gelatine had taken place and before the addition of lecithin. After the solubilisation of gelatine the mixture was stirred for 20 minutes and the temperature of the reactor was raised to 75-80° C. and 160 g of the stabilising agent were slowly and continuously added during approximately 5 minutes.

The following compositions were manufactured following this process:

| Exam- | Stabilising agent | Stabilising agent | Melting range |
|---|---|---|---|
| ple | (Tradename) | (Chemical nature) | (° C.) |
| 2 | Cutine HR | Hydrogenated castor oil | 87-88 |
| 3 | Compritol 888 ATO | Glyceryl behenate | 71.4-72.2 |
| 4 | Akofine NF | Hydrogenated cottonseed | 63.4-63.9 |
| | | oil | |
| 5 | Estol 3745 GDS T2 | Glyceryl diestearate 80 | 59.0-59.7 |
| 6 | Gelucire 50/13 | Stearoyl macrogol-32 | 50.3-51.0 |
| | | glycerides | |
| 7 | Gelucire 44/14 | Lauryl macrogol-32 | 43.6-44.2 |
| | | glycerides | |

To evaluate the contribution of the stabilising agent, the compositions of examples 1 to 7 were subjected to the “removal from blister test” described above with the following results.

| Example | Removal from Blister Test |
|---|---|
| 1 | Failed |
| 2 | Failed |
| 3 | Failed |
| 4 | Failed |
| 5 | Passed |
| 6 | Passed |
| 7 | Passed |

1. A non-tabletted, individually dosed administration form comprising a composition comprising at least one pharmaceutically active substance dissolved or dispersed within a matrix material, at least one stabilising agent, and at least one water-soluble alcohol and/or water as a solvent;
wherein the matrix material comprises a mixture comprising at least 0.2% by weight of a gelatine;
wherein the composition is plastic at elevated temperature;
wherein:
a. the at least one stabilising agent is chosen from (i) esters of glycerine and fatty acids; and (ii) products resulting from the alcoholysis/esterification reaction of said esters of glycerine and fatty acids with polyethylenglycols;
b. the at least one stabilising agent has a melting point ranging from 42° C. to 63° C.; and
c. water is optionally present in an amount not greater than 46% by weight of the composition.

2. A non-tabletted, individually dosed administration form according to claim 1, wherein the at least one stabilising agent is present in an amount greater than 1% by weight of the composition.

3. A non-tabletted, individually dosed administration form according to claim 1, wherein the administration form is packaged in blisters or cavities shaped from films.

4. A non-tabletted, individually dosed administration form according to claim 1, wherein the administration form comprises more than 18% by weight of at least one pharmaceutically active substance.

5. A non-tabletted, individually dosed administration form according to claim 1, wherein the administration form comprises an antacid.

6. A non-tabletted, individually dosed administration form according to claim 1, wherein the administration form comprises at least 10% by weight of the composition of at least one water-soluble alcohol and/or water as a solvent.

7. A non-tabletted, individually dosed administration form according to claim 6, wherein the administration form comprises more than 46% by weight of the composition of at least one water-soluble alcohol.

8. A non-tabletted, individually dosed administration form according to claim 1, wherein the composition comprises water.

9. A non-tabletted, individually dosed administration form according to claim 1, wherein the composition does not comprise edible gums.

10. A process for producing a non-tabletted, individually dosed administration form comprising:
forming a composition comprising at least one pharmaceutically active substance dispersed or dissolved within a matrix material, at least one stabilising agent and at least one water-soluble alcohol and/or water as a solvent,
wherein the matrix material comprises a mixture comprising at least 0.2% by weight of a gelatine;
where the composition is plastic at elevated temperature;
keeping the composition above 37° C.;
transferring the composition, when it is fluid, into a heated dosing apparatus;
discharging the composition onto a shaped substrate, such that a constant quantity of the fluid formulation material is thereby dosed onto the substrate;
cooling the composition; and
optionally sealing the substrate containing the composition;
wherein water is optionally present in an amount not greater than 46% by weight of the composition;
wherein the at least one stabilising agent is chosen from (i) esters of glycerine and fatty acids; and (ii) products resulting from the alcoholysis/esterification reaction of said esters with polyethyleneglycols, and
wherein the at least one stabilising agent has a melting point in the range of 42° C. to 63° C.

11. A process according to claim 10, wherein the at least one stabilising agent is present in an amount greater than 1% by weight of the composition.

12. A process according to claim 10, wherein the composition comprises more than 18% by weight of at least one pharmaceutically active substance.

13. A process according to claim 10, wherein the at least one pharmaceutically active substance comprises an antacid.

14. A process according to claim 10, wherein the composition comprises water.

15. A process according to claim 10, wherein the composition comprises at least 10% by weight of the composition of at least one water-soluble alcohol and/or water as a solvent.

16. A process according to claim 10, wherein the composition comprises more than 46% by weight of the composition of at least one water-soluble alcohol.

17. A process according to claim 10, wherein the composition does not comprise edible gums.

18. A process according to claim 10, wherein the shaped substrate is a cavity or blister, and wherein the cavity or blister comprises a material chosen from PVC, PVDC, PP, Aclar and laminates.

19. A non-tabletted, individually dosed administration form obtainable by the process of claim 10.

20. A method for facilitating the removal of a composition from a blister or cavity, comprising adding at least one stabilising agent to the composition;
wherein the at least one stabilising agent is chosen from (i) esters of glycerine and fatty acids and (ii) products resulting from the alcoholysis/esterification reaction of such esters with polyethyleneglycols,
wherein the at least one stabilising agent has a melting point in the range of 42° C. to 63° C.;
wherein the composition comprises at least one pharmaceutically active substance dispersed or dissolved within a matrix material;
wherein the matrix material comprises a mixture of gelatine and at least one water-soluble alcohol and/or water as a solvent, and
wherein the composition is plastic at elevated temperature.

21. A method according to claim 20, wherein the at least one stabilising agents is present in the composition in an amount greater than 1% by weight of the composition.

22. (canceled)

23. A non-tabletted, individually dosed administration form according to claim 8, wherein the administration form comprises water in an amount exceeding 1% wt. of the composition.

24. A process according to claim 14, wherein the composition comprises water in an amount exceeding 1% wt. of the composition.

25. A process according to claim 18, wherein the cavity or blister comprises OPA-Aluminium-PVC.
